(19) United States
(12) Patent Application Publication
(10) Pub. No.: US 2025/0163451 A1
(43) Pub. Date: May 22, 2025

(54) RESISTANCE GENE TACAT TO FUSARIUM CROWN ROT OF WHEAT AND APPLICATION THEREOF

(71) Applicant: Henan Agricultural University, Zhengzhou (CN)

(72) Inventors: Feng Chen, Zhengzhou (CN); Xia Yang, Zhengzhou (CN); Dan Pei, Zhengzhou (CN); Xiaodong Yu, Zhengzhou (CN); Yan Yan, Zhengzhou (CN); Yan Ren, Zhengzhou (CN); Ning Zhang, Zhengzhou (CN); Lei Zhao, Zhengzhou (CN)

(21) Appl. No.: 18/827,889

(22) Filed: Sep. 9, 2024

(30) Foreign Application Priority Data
Nov. 20, 2023 (CN) 202311547240.9

(51) Int. Cl. C12N 15/82; C12Q 1/6895

(52) U.S. Cl. CPC C12N 15/8282; C12Q 1/6895; C12Q 2600/13; C12Q 2600/156

(57) ABSTRACT

A resistance gene TaCAT to FCR of wheat and an application thereof are provided, which relates to the field of biological genetic engineering technologies. The nucleotide sequence of a genome where the TaCAT gene is located, the nucleotide sequence of a cDNA, the nucleotide sequence of a CDS and a coded protein sequence are respectively as shown in SEQ ID NO: 1, SEQ ID NO: 2, SEQ ID NO: 3 and SEQ ID NO: 4. An InDel marker is developed for identifying different allelotypes of the TaCAT gene. An application of the TaCAT gene for preparation of transgenic plant cells and plant breeding is provided. The molecular genetic basis of the resistance to FCR is disclosed, an important role is played in improving the resistance to FCR by utilizing a genetic engineering technology, and a new way is provided for breeding a new wheat variety with disease-resistance and safety.

### CROSS-REFERENCE TO RELATED APPLICATION

This application claims priority to Chinese Patent Application No. 202311547240.9, filed on Nov. 20, 2023, which is herein incorporated by reference in its entirety.

### TECHNICAL FIELD

The disclosure relates to the field of biological genetic engineering technologies, and more particularly to a resistance gene TaCAT to Fusarium crown rot of wheat and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24086TBYX-USP1-SL.xml. The XML file is 21,316 bytes; is created on Aug. 9, 2024; and is being submitted electronically via patent center.

### BACKGROUND

Fusarium crown rot (FCR) of wheat is a typical soil-borne disease, which was first reported for similar symptoms in Australia in 1940 by Mcknight et al (McKnight T, Hart J, Some field observations on crown rot disease of wheat caused by Fusarium graminearum, Queensland Journal of Agricultural and Animal Sciences, 1966, 23:373-378). At present, the FCR of wheat has spread to become a global disease, which is widely distributed around the world, including North America, South America, Europe, Africa, Mediterranean coastal countries, most Asian countries, New Zealand and Australia in Oceania, etc. In China, the FCR of wheat caused by Fusarium pseudograminearum was first reported in 2012.

The FCR of wheat not only causes serious yield and economic losses during its occurrence, but also produces multiple toxins and secondary metabolites, such as nivalenol (NIV), deoxynivalenol (DON) and zearalenone (ZEN) (Yang et al., Investigation and genome-wide association study for Fusarium crown rot resistance in Chinese common wheat, BMC Plant Biology, Apr. 23, 2019, 19: 153). On the one hand, these substances can be pathogenic factors of pathogens to damage plants, on the other hand, these substances can contaminate host plants and affect the safety of plant products, processed foods and feeds. NIV and DON are potential carcinogens, which can cause serious harm to health of human and livestock.

Due to scarcity of disease-resistant resources and narrow genetic background of current varieties, a wheat variety with immunity or high resistance to the FCR has not yet been identified. At present, researches on the FCR of wheat are mostly focused on collection of pathogens, identification of material resistance and preliminary mapping of genes. Up to now, almost all 21 chromosomes of hexaploid wheat have been reported to contain quantitative trait loci (QTL) for resistance to the FCR, and the QTL that can be repeatedly detected are located on chromosomes 2DL, 3BL, 4BS and 5DS, but these QTL often contain only partial resistance or unstable resistance (Yang et al., A loss-of-function of the dirigent gene TaDIR-B1 improves resistance to Fusarium crown rot in wheat, Plant Biotechnology Journal, May 2021, 19: 866-868). In addition, the reported genes related to resistance to the FCR of wheat include Fhb7 (Wang et al., Horizontal gene transfer of Fhb7 from fungus underlies Fusarium head blight resistance in wheat, Science, Apr. 9, 2020,368:5435), TaDIR-4B (Yang et al., A loss-of-function of the dirigent gene TaDIR-B1 improves resistance to Fusarium crown rot in wheat, Plant Biotechnology Journal, May 2021, 19:866-868) and TaCWI-B1 (Lv et al., A cell wall invertase modulates resistance to fusarium crown rot and sharp eyespot in common wheat, Journal of Integrative Plant Biology, July 2023, 65:1814-1825). These genes play an important role in resistance of wheat to the FCR infection, but the effect size and stability of the genes need to be further verified.

Due to a lack of recognized identification standards for the FCR of wheat, generally poor resistance of wheat varieties, and significant genetic background limitations, the discovery and development of resistance genes to the FCR of wheat has been slow. Therefore, expanding a scope of germplasm resource screening, discovering and utilizing excellent disease-resistant genes, and exploring regulatory pathways involved in the disease-resistant genes are of great significance to aggregation and utilization of the resistance genes to the FCR of wheat.

### SUMMARY

A purpose of the disclosure is to provide a resistance gene TaCAT to FCR of wheat and an expression product thereof, as well as primers, a silencing vector and a plant expression vector for detecting genotypes of the FCR of wheat, and to apply the resistance gene TaCAT to the FCR of wheat and the expression product thereof, as well as the primers, the silencing vector and the plant expression vector to prevention and breeding of the FCR of wheat, to thereby provide more selections for breeding for resistance to the FCR of wheat.

In order to solve the above technical problems, the disclosure adopts the following technical solutions.

1. The disclosure provides a TaCAT gene related to the FCR of wheat, the nucleotide sequence of a genome where the TaCAT gene is located is as shown in SEQ ID NO: 1, the nucleotide sequence of a full-length complementary deoxyribonucleic acid (cDNA) of the TaCAT gene is as shown in SEQ ID NO: 2, and the nucleotide sequence of a coding sequence (CDS) of the TaCAT gene is as shown in SEQ ID NO: 3.

2. The disclosure provides a protein coded by the TaCAT gene related to the FCR of wheat, and the amino acid sequence of the protein coded by the TaCAT gene is as shown in SEQ ID NO: 4.

3. The disclosure further provides a specific primer pair for detecting the TaCAT gene related to the FCR of wheat, and the specific primer pair includes: an upstream primer with the nucleotide sequence as shown in SEQ ID NO: 5, and a downstream primer with the nucleotide sequence as shown in SEQ ID NO: 6.

A method for detecting an allelotype of the FCR of wheat by using the above specific primer pair includes:
step 1, extracting genomic DNA of a to-be-detected wheat; and
step 2, taking the genomic DNA of the to-be-detected wheat as a template, performing a polymerase chain reaction (PCR) amplification with the upstream primer with the nucleotide sequence as shown in SEQ ID NO: 5, and the downstream primer with the nucleotide sequence as shown in SEQ ID NO: 6, to obtain an amplification product as shown in SEQ ID NO: 1, and detecting the allelotype of the Fusarium crown rot of wheat based on the amplification product as shown in SEQ ID NO: 1; and
where a resistant and susceptible material has an insertion or deletion (InDel) of 215 base pairs (bp) starting from a 654^{th} position after a start codon adenine-thymine-guanine (ATG) in the gene sequence as shown in SEQ ID NO: 1, and a genotype of the amplification product is determined according to the InDel; and a genotype corresponding to an insertion type amplification product is named as TaCAT-6Aa, and a genotype corresponding to a deletion type amplification product is named as TaCAT-6Ab.

The phenotype identification results show that a plant corresponding to the genotype of TaCAT-6Aa is severely affected and has a high disease index, and resistance of the plant corresponding to the genotype of TaCAT-6Aa to the FCR of wheat is lower than resistance of a plant corresponding to the genotype of TaCAT-6Ab to the FCR of wheat. The plant corresponding to the genotype of TaCAT-6Ab has mild disease incidence and a low disease index, and the resistance of the plant corresponding to the genotype of TaCAT-6Ab to the FCR of wheat is higher than the resistance of the plant corresponding to the genotype of TaCAT-6Aa to the FCR of wheat. Therefore, the TaCAT gene plays a crucial role in the resistance to the FCR of wheat.

Alternatively, in step 2, an InDel marker is developed according to the insertion or deletion fragment to detect different genotypes of the TaCAT gene. A differential site-specific primer pair includes: an upstream primer with the nucleotide sequence as shown in SEQ ID NO: 7, and a downstream primer with the nucleotide sequence as shown in SEQ ID NO: 8.

The specific InDel marker is used to amplify DNA from different materials. A genotype of a wheat variety/strain with an amplification product of 1054 bp is named as TaCAT-6Aa, a genotype of a wheat variety/strain with an amplification product of 839 bp is named as TaCAT-6Ab (FIG. 4A), and there is a significant difference in the disease resistance ability of plants corresponding to the two genotypes.

4. The disclosure further designs a virus-induced gene silencing (VIGS) vector of the TaCAT gene.

Primer sequences for amplifying a silencing fragment include: an upstream primer with the nucleotide sequence as shown in SEQ ID NO: 9, and a downstream primer with the nucleotide sequence as shown in SEQ ID NO: 10. The silencing fragment of PCR amplification can be seamlessly cloned and connected between restriction enzyme cutting sites PacI and NotI of a γ-PDS-as vector to obtain a recombinant plasmid.

The recombinant plasmid is further linearized and transcribed in vitro to obtain RNA virus, which is inoculated into wheat seedling leaves to silence the TaCAT gene (a blank control and a whitening positive control are set up), to thereby verify the function of the TaCAT gene.

5. The disclosure further designs a plant expression vector containing the TaCAT gene, and the above recombinant vector (i.e., the recombinant plasmid) can be used to construct recombinant bacteria.

The plant expression vector used is specifically a binary Agrobacterium vector or a plant microprojectile bombardment vector, and the recombinant vector is obtained by inserting the nucleic acid sequence as shown in SEQ ID NO: 3 between restriction enzyme cutting sites of the plant expression vector.

The plant expression vector can also include a 3′-untranslated region of a foreign gene (also referred to as exogenous gene), that is, the plant expression vector includes a polyadenylic acid signal and other DNA fragments involved in mRNA processing or gene expression. The polyadenylic acid signal can guide addition of polyadenylic acid to a 3′ end of a mRNA precursor. For example, the Agrobacterium tumefaciens induction (tumor-inducing abbreviated as Ti) plasmid gene and the untranslated region transcribed at the 3′ end of the plant gene (such as soybean storage protein gene) have a similar function. When the gene is used to construct a recombinant plant expression vector, any enhanced promoter or constitutive promoter can be added before transcribing a start nucleotide, and the enhanced promoter or the constitutive promoter can be used alone or in combination with other plant promoters. In addition, when the gene of the disclosure is used to construct the plant expression vector, an enhancer can also be used, and the enhancer includes a transcription enhancer or a translation enhancer. The enhancer regions can be ATG start codons or start codons in adjacent regions, but must be the same as a reading frame of the coding sequence to ensure a correct translation of the entire sequence. In order to facilitate the identification and screening of transgenic plant cells or transgenic plants, the plant expression vector used can be processed and modified, such as adding enzymes or luminescent groups (luciferase gene, β-glucuronidase abbreviated as GUS gene, etc.) that can produce color changes through coding, antibiotic markers (kanamycin markers, gentamicin markers, etc.) with resistance, or chemical agent resistance marker genes (herbicide resistance genes, etc.) that can be expressed in plants. For considering the safety of the transgenic plants, no selective marker genes can be added, and transformed plants can be directly screened with appropriate disease pressure.

The disclosure further provides an application method of the TaCAT gene, including: breeding a wheat variety resistant to the FCR by using the TaCAT gene. That is, the TaCAT gene is transformed into a target plant to obtain a transgenic plant for adjusting resistance to the FCR. The expression vector carrying the TaCAT gene can transform plant cells or tissues by using conventional biological methods such as Ti plasmid, rhizobium-inducible (Ri) plasmid (also referred to root-inducing plasmid), plant virus vector, DNA transformation, microinjection, electroporation, Agrobacterium-mediated transformation, gene gun, etc., and the transformed plant tissues are cultivated into a plant for subsequent research.

The target plant can be either a monocot or a dicot. The monocot can be wheat, such as Kenong 199 (KN199, cultivated by Institute of Genetics and Developmental Biology, Chinese Academy of Sciences in 2006, approval number: national approved wheat variety 2006017).

Compared to the related art, main beneficial technical effects of the disclosure are as follows.

1. The disclosure has identified a new gene related to the FCR of wheat, which is named TaCAT, the gene is located on a chromosome 6A of the wheat and the protein expressed by the gene can regulate resistance of wheat to the FCR.

2. The disclosure clarifies the DNA sequence, CDS sequence and the coded protein sequence of the TaCAT gene related to the FCR of wheat, which lays a technical foundation for application practice of breeding a wheat variety resistant to the FCR in the future.

3. The specific primers in the disclosure can be directly used for the detection of different alleles of the FCR of wheat, which is beneficial to the screening and breeding of a disease-resistant wheat variety and can effectively save time and resources.

4. The disclosure uses the VIGS technology to silence the TaCAT gene related to the FCR of wheat, to thereby verify a role of the TaCAT gene in breeding the wheat variety resistant to the FCR.

5. The disclosure uses the transgenic technology to transform the resistance gene TaCAT to the FCR of wheat into a common wheat for overexpression for the first time, to thereby verify the role of the TaCAT gene in breeding the wheat variety resistant to the FCR.

6. The disclosure helps to reveal a molecular genetic basis of the FCR of wheat. Meanwhile, the disclosure plays an important role in detecting and improving wheat varieties by using a genetic engineering technology, and provides a new way to breed disease-resistant and safe new wheat varieties.

### BRIEF DESCRIPTION OF DRAWINGS

FIG. 1 illustrates a schematic diagram of a genome-wide association study (GWAS) combined with a whole exome sequencing (WES) to locate resistance regions. Specifically, a represents the GWAS; b represents the WES; c represents a co-located segment; and d represents polymorphic gene within the co-located segment.

FIG. 2A illustrates a schematic diagram of differentially expressed genes within a localization segment excavated by a bulking transcriptome. Specifically, FCR-RP represents a material in a resistance pool to FCR; FCR-SP represents a material in a susceptible pool to the FCR; i represents a bacterial inoculation treatment; and n represents no bacterial inoculation treatment.

FIG. 2B illustrates a schematic diagram of differentially expressed proteins within a localization segment excavated by a bulking proteome. Specifically, PR represents a resistance parent; and PS represents a susceptible parent.

FIG. 3A illustrates a schematic structural diagram of a TaCAT gene in a resistant and susceptible material.

FIG. 3B illustrates a schematic diagram of an analysis of different haplotypes of the TaCAT gene.

FIG. 3C illustrates a schematic diagram of corresponding phenotypes of the different haplotypes of the TaCAT gene.

FIG. 4A illustrates a detecting schematic diagram of an InDel marker of the TaCAT gene in a part of families in a JY population.

FIG. 4B illustrates a schematic diagram of a verification result of the InDel marker of the TaCAT gene in descendant populations.

FIG. 5 illustrates a schematic diagram of a relative expression level of the TaCAT gene in a VIGS experiment.

FIG. 6A illustrates a schematic diagram of effects of the TaCAT gene silenced by the VIGS of wheat treated with different materials.

FIG. 6B illustrates a schematic diagram of disease indexes of wheat treated with different materials after silencing the TaCAT gene.

FIG. 7A illustrates a schematic diagram of a cat1 mutant site.

FIG. 7B illustrates a schematic comparative diagram of phenotypes of resistance to FCR of wheat of a Kronos3868 positive mutant (i.e., the cat1) and a wide-type Kronos in a seedling stage.

FIG. 7C illustrates a schematic comparative diagram of phenotypes of resistance to FCR of wheat of the cat1 and the wide-type Kronos in an adult plant stage.

FIG. 7D illustrates a schematic comparative diagram of disease indexes of the cat1 and the wide-type Kronos in the seedling stage and the adult plant stage.

FIG. 8 illustrates a schematic diagram of a graph of an overexpression vector pLGY-OE3.

FIG. 9 illustrates a schematic diagram of relative expression levels of transgenic lines (TaCAT-OX1 and TaCAT-OX2) with TaCAT gene overexpression.

FIG. 10A illustrates a schematic comparative diagram of phenotypes of resistance to FCR of wheat of the transgenic lines (TaCAT-OX1 and TaCAT-OX2) with TaCAT gene overexpression and a wide-type KN199 in the seedling stage.

FIG. 10B illustrates a schematic comparative diagram of phenotypes of resistance to FCR of wheat of the transgenic lines (TaCAT-OX1 and TaCAT-OX2) with TaCAT gene overexpression and the wide-type KN199 in the adult plant stage.

FIG. 10C illustrates a schematic comparative diagram of disease indexes of the transgenic lines (TaCAT-OX1 and TaCAT-OX2) with TaCAT gene overexpression and the wide-type KN199 in the seedling stage and the adult plant stage.

### DETAILED DESCRIPTION OF EMBODIMENTS

The disclosure is described in more detail below through embodiments, to facilitate understanding of technical solutions of the disclosure, but is not intended to limit a scope of protection of the disclosure.

Unless otherwise specified, instruments and equipment involved in the following embodiments are all conventional instruments and equipment. Phenotypic identification experiments in the following embodiments are set up for three repeated experiments, and results are averaged. Primer synthesis and sequencing are completed by Sangon Biotech (Shanghai) Co., Ltd.

The involved wheat materials include: a natural population consisting of 243 main varieties promoted in the Huanghuai wheat region in recent years (Table 1), two F₄ generation recombinant inbred lines (RIL) populations (including a JY population and a JZ population), Jinmai 1 (JM1, cultivated by Shanxi Sanyuan Golden Seed Zhongye Technology Co., Ltd. and Xianyang Agricultural Science Research Institute cultivateShaanxi Sanyuan Golden Seed Zhongye Technology Co., Ltd. and Xianyang Agricultural Science Research Institute, approval number: Shanxi approved wheat variety: 2014006), a Kronos mutant (mutated by Jorge Dubcovsky, gene ID: TraesCS6A02G041700.1) and KN199.

A wheat 660 kilo (K) single nucleotide polymorphism (SNP) array (Sun et al., The Wheat 660K SNP array demonstrates great potential for marker-assisted selection in polyploid wheat, Plant Biotechnology Journal, June 2020, 18:1354-1360) covering a whole genome is used for genotyping on the natural population, and the natural population is used for GWAS.

The two F₄ generation RIL populations are constructed by hybridizing a disease-resistant variety JM1 with susceptible varieties Yunong 805 (YN805, cultivated by Henan agricultural university, approval number: Henan approved wheat variety: 20180023) and Zhengmai 082 (ZM082, cultivated by Henan Academy of Agricultural Sciences, approval number: Henan approved wheat variety 20220055), respectively. The two F₄ generation RIL populations are used for WES, bulking transcriptome sequencing, and bulking proteome sequencing.

The JM1 has genotype and phenotype resistant to FCR of wheat, and used for a VIGS experiment to verify a disease-resistance effect of a TaCAT gene;

The Kronos mutant includes a wild-type Kronos-WT and Kronos3868 (the TaCAT gene premature termination mutant), and used to verify the disease resistance effect of the TaCAT gene.

The KN199 is a susceptible variety for the FCR of wheat, and is used as an overexpression transgenic receptor material to verify the disease-resistance function of the TaCAT gene.

TABLE 1
Details of population materials used for GWAS

| Number | Name | Number | Name | Number | Name | Number | Name |
|---|---|---|---|---|---|---|---|
| 1 | Tianning 18 | 2 | Zhongxin 18 | 3 | Jiamai 99 | 4 | Keyu 368 (KY368) |
| | (TN18) | | (ZX18) | | (JM99) | | |
| 5 | Zongmai 108 | 6 | Bainong 1309 | 7 | Hemai 181 | 8 | Taixue 30 (TX30) |
| | (ZM108) | | (BN1309) | | (HM181) | | |
| 9 | Qunximai | 1110 | Fengtian 18 | 11 | Heyu 1 (HY1) | 12 | Shengmai 102 |
| | (QXM11) | | (FT18) | | | | (SM102) |
| 13 | Xinmai 68 | 14 | Zhengmai 1869 | 15 | Hongmai 618 | 16 | Fengdecunmai 20 |
| | (XM68) | | (ZM1869) | | (HM618) | | (FDCM20) |
| 17 | Xinyoumai 2 | 18 | Jiamai 6 (JM6) | 19 | Fanmai 533 | 20 | Zhengmai 518 |
| | (XYM2) | | | | (FM533) | | (ZM518) |
| 21 | Xingnong 168 | 22 | Yanmai 9719 | 23 | Luo 1807 | 24 | Yanfeng 712 |
| | (XN168) | | (YM9719) | | (L1807) | | (YF712) |
| 25 | Zimai 627 | 26 | Yunong 805 | 27 | Zhengxin 758 | 28 | Gengmai 256 |
| | (ZM627) | | (YN805) | | (ZX758) | | (GM256) |
| 29 | Yanke 316 | 30 | Fumai 188 | 31 | Lunxuan 162 | 32 | Jingjiumai 11 |
| | (YK316) | | (FM188) | | (LX162) | | (JJM11) |
| 33 | Yunong 804 | 34 | Gengmai 237 | 35 | Jinmai 1 (JM1) | 36 | Liming 28 (LM28) |
| | (YN804) | | (GM237) | | | | |
| 37 | Qinmai 158 | 38 | Junmai 118 | 39 | Caiyuan 1 (CY1) | 40 | Junhe 183 (JH183) |
| | (QM158) | | (JM118) | | | | |
| 41 | Ximai 505 | 42 | Kaimai 26 | 43 | Zhoukang 918 | 44 | Zhengnong 06118 |
| | (XM505) | | (KM26) | | (ZK918) | | (ZN06118) |
| 45 | Nongda 399 | 46 | Zhongying 012 | 47 | Zhouyumai 36 | 48 | Jinying 18 (JY18) |
| | (ND399) | | (ZY012) | | (ZYM36) | | |
| 49 | Zhengmai 082 | 50 | Zhengmai 516 | 51 | Xuke 877 | 52 | Zhongkenmai 7 |
| | (ZM082) | | (ZM516) | | (XK877) | | (ZKM7) |
| 53 | Chuangxing 6 | 54 | Luyuan 502 | 55 | Zhongfengmai | 256 | Hengmai 18 |
| | (CX6) | | (LY502) | | (ZFM2) | | (HM18) |
| 57 | Neimai | 658 | Fengbao 8 (FB8) | 59 | Tianle 6 (TL6) | 60 | Xinmai 37 (XM37) |
| | (NM6) | | | | | | |
| 61 | Hemai | 662 | Junmai 667 | 63 | Nongfeng 11164 | | Wenliang 1 (WL1) |
| | (HM6) | | JM667) | | (NF111) | | |
| 65 | Pingan 0602 | 66 | Huayu 3568 | 67 | Guangtai 369 | 68 | Yunong 169 |
| | (PA0602) | | (HY3568) | | (GT369) | | (YN169) |
| 69 | Zhonglemai | 970 | Jinsui 8 (JS8) | 71 | Jingkemai 6 | 72 | Xinxuan 17 |
| | (ZLM9) | | | | (JKM6) | | (XX17) |
| 73 | TH161 | 74 | Jinmai 108 | 75 | Jinyan 5 (JY5) | 76 | Huayu 126 |
| | | | (JM108) | | | | (HY126) |
| 77 | Baiqiang 1201 | 78 | Yimai 8 (YM8) | 79 | Hefeng 3 (HF3) | 80 | Lunxuan 163 |
| | (BQ121) | | | | | | (LX163) |
| 81 | Yunong 019 | 82 | Zhongle 8 (ZL8) | 83 | Ruisen 218 | 84 | Fanning 3 (FN3) |
| | (YN019) | | | | (RS218) | | |
| 85 | Hemai 2 | 86 | Xuke 732 | 87 | Jinmai 14 | 88 | Haozhuangjia 1 |
| | (HM2) | | (XK732) | | (JM14) | | (HZJ1) |
| 89 | Xuyou 46 | 90 | Hanmai 457 | 91 | Fengdecunmai | 192 | Hangmai 8 (HM8) |
| | (XY46) | | (HM457) | | 19 (FDCM19) | | |
| 93 | L668 | 94 | Qiangmai 29 | 95 | Luyan 260 | 96 | Yanhao 306 |
| | | | (QM29) | | (LY260) | | (YH306) |
| 97 | Caizhi 204 | 98 | Zhengpinmai 24 | 99 | Luomai 2 (LM2) | 100 | Anyumai 18 |
| | (CZ204) | | (ZPM24) | | | | (AYM18) |
| 101 | Wenyu 019 | 102 | Taipingyuan 18 | 103 | Jingyumai 1 | 104 | Luomai 718 |
| | (WY019) | | (TPY18) | | (JYM1) | | (LM718) |
| 105 | Yanmai 26 | 106 | Junmai 188 | 107 | Tianmai 119 | 108 | Fengmai 53 |
| | (YM26) | | (JM188) | | (TM119) | | (FM53) |
| 109 | Danmai 108 | 110 | Xinmai 38 | 111 | Changshengmai | 112 | Defeng 108 |
| | (DM108) | | (XM38) | | 1 (CSM1) | | (DF108) |
| 113 | Shaomai 25 | 114 | Tongfeng 736 | 115 | Meng 615 | 116 | Boyu 866 (BY866) |
| | (SM25) | | (TF736) | | (M615) | | |
| 117 | Bainong 1306 | 118 | Jiamai 8 (JM8) | 119 | Xianhong 169 | 120 | Yanmai 888 |
| | (BN1306) | | | | (XH169) | | (YM888) |
| 121 | Shengyuan 928 | 122 | Jun 5366 (J5366) | 123 | Zhengnong 5222 | 124 | Xuke 682 (XK682) |
| | (SY928) | | | | (ZN5222) | | |
| 125 | Xinzhi 519 | 126 | Xuyan 2 (XY2) | 127 | Hangmai 6 | 128 | Yunong 1235 |
| | (XZ519) | | | | (HM6) | | (YN1235) |
| 129 | Fanmai 536 | 130 | Yufeng 1366 | 131 | Xinmai 12 | 132 | Xunong 618 |
| | (FM536) | | (YF1366) | | (XM12) | | (XN618) |
| 133 | Ningnong 718 | 134 | Yuyan 168 | 135 | Shunmai 8 | 136 | Aomai 18 (AM18) |
| | (NN718) | | (YY168) | | (SM8) | | |
| 137 | Pumai 27 | 138 | Tianlaoda 3 | 139 | Xingyu 7 (XY7) | 140 | Xinong 18 (XN18) |
| | (PM27) | | (TLD3) | | | | |
| 141 | Yongfengnong | 142 | Guangtai 213 | 143 | Qiule 2126 | 144 | Nongfeng 8210 |
| | 2 (YFN2) | | (GT213) | | (QL2126) | | (NF8210) |
| 145 | Saidemai 7 | 146 | Dongfanghongmai | 147 | Jinmai 18 | 148 | Jimai 210 (JM210) |
| | (SDM7) | | 6 (DFH6) | | (JM18) | | |
| 149 | Kaimai 27 | 150 | Jinmai 109 | 151 | Hengda 58 | 152 | Maifeng 9 (MF9) |
| | (KM27) | | (JM109) | | (HD58) | | |
| 153 | Jinchengmai 10 | 154 | Xianmai 15 | 155 | Yanmai 988 | 156 | Xianyuan 988 |
| | (JCM10) | | (XM15) | | (YM988) | | (XY988) |
| 157 | Lunxuan 169 | 158 | Xinmai 8 (XM8) | 159 | Wenmai 29 | 160 | Wenyuan 0528 |
| | (LX169) | | | | (WM29) | | (WY0528) |
| 161 | Jinwoye 1 | 162 | Changmai 13 | 163 | Kun 169 (K169) | 164 | SM110 |
| | (JWY1) | | (CM13) | | | | |
| 165 | Bainong 219 | 166 | Sanhe 1 (SH1) | 167 | Xiza 5 (XZ5) | 168 | Yufeng 6 (YF6) |
| | (BN219) | | | | | | |
| 169 | Taipingyuan | 170 | Songmai 518 | 171 | Shenzhou 209 | 172 | Yunong 99 |
| | 007 (TPY007) | | (SM518) | | (SZ209) | | (YN99) |
| 173 | Shangmai 162 | 174 | Junmai 612 | 175 | Jiyanmai 10 | 176 | Aifeng 338 |
| | (SM162) | | (JM612) | | (JYM10) | | (AF338) |
| 177 | Liangmai 958 | 178 | Fengmai 52 | 179 | Chuangmai 11 | 180 | Wanmai 99 |
| | (LM958) | | (FM52) | | (CM11) | | (WM99) |
| 181 | Xinhuamai 818 | 182 | Zhumai 706 | 183 | Taifeng 11 | 184 | Luomai 32 (LM32) |
| | (XHM818) | | (ZM706) | | (TF11) | | |
| 185 | Zhongmai 10 | 186 | Jinchengmai 12 | 187 | Chuangxin 106 | 188 | Huaichuan 361 |
| | (ZM10) | | (JCM12) | | (CX106) | | (HC361) |
| 189 | Xianmai 522 | 190 | Jinfeng 216 | 191 | Jinzhan 638 | 192 | Xiangmai 1123 |
| | (XM522) | | (JF216) | | (JZ638) | | (XM1123) |
| 193 | Lunxuan 167 | 194 | Xuke 158 | 195 | Chuangxin 116 | 196 | Zhengda 101 |
| | (LX167) | | (XK158) | | (CX116) | | (ZD101) |
| 197 | Jiamai 329 | 198 | Luomai 166 | 199 | Fanning 1 (FN1) | 200 | Kelinmai 969 |
| | (JM329) | | (LM166) | | | | (KLM969) |
| 201 | Bomai 118 | 202 | Wohua 066 | 203 | Minfeng 296 | 204 | Zimai 615 |
| | (BM118) | | (WH066) | | (MF296) | | (ZM615) |
| 205 | Xuanmai 6 | 206 | Weimin 208 | 207 | Yingmai 182 | 208 | Shenhua 208 |
| | (XM6) | | (WM208) | | (YM182) | | (SH208) |
| 209 | Jumai 66 | 210 | Yufeng 2 (YF2) | 211 | Yanmai 68 | 212 | Yumai 117 |
| | (JM66) | | | | (YM68) | | (YM117) |
| 213 | Liangyuan A6 | 214 | Mengnong 1 | 215 | Zhenmai 5 | 216 | Xinxuan 16 |
| | (LYA6) | | (MN1) | | (ZM5) | | (XX16) |
| 217 | Lifu 05 (LF05) | 218 | Fanyumai 18 | 219 | Yingman 180 | 220 | Xianmai 521 |
| | | | (FYM18) | | (YM180) | | (XM521) |
| 221 | Jiangmai 816 | 222 | Danmai 118 | 223 | Tianmin 688 | 224 | Shuanmai 299 |
| | (JM816) | | (DM118) | | (TM688) | | (SM299) |
| 225 | Xuyan 3 (XY3) | 226 | Zhengda 3087 | 227 | Zhaofeng 668 | 228 | Fengmai 10 |
| | | | (ZD3087) | | (ZF668) | | (FM10) |
| 229 | Chuangxin 26 | 230 | Yufeng 1 (YF1) | 231 | Neile 268 | 232 | Pingnongyan 3 |
| | (CX26) | | | | (NL268) | | (PNY3) |
| 233 | Hongtaiyang 2 | 234 | Tianlaoda 1 | 235 | Xinyanmai 98 | 236 | Hongmai 186 |
| | (HTY2) | | (TLD1) | | (XYM98) | | (HM196) |
| 237 | Huayan | 328238 | Huimai | 216239 | Nongda 2018240 | | Jiyanmai 7 (JYM7) |
| | (HY328) | | (HM216) | | (ND2018) | | |
| 241 | Jinfeng 205 | 242 | Taihemai 3 | 243 | Zhengke 6 (ZK6) | | |
| | (JF205) | | (THM3) | | | | |

### Embodiment 1 Mapping and Acquisition of the TaCAT Gene Related to the FCR of Wheat

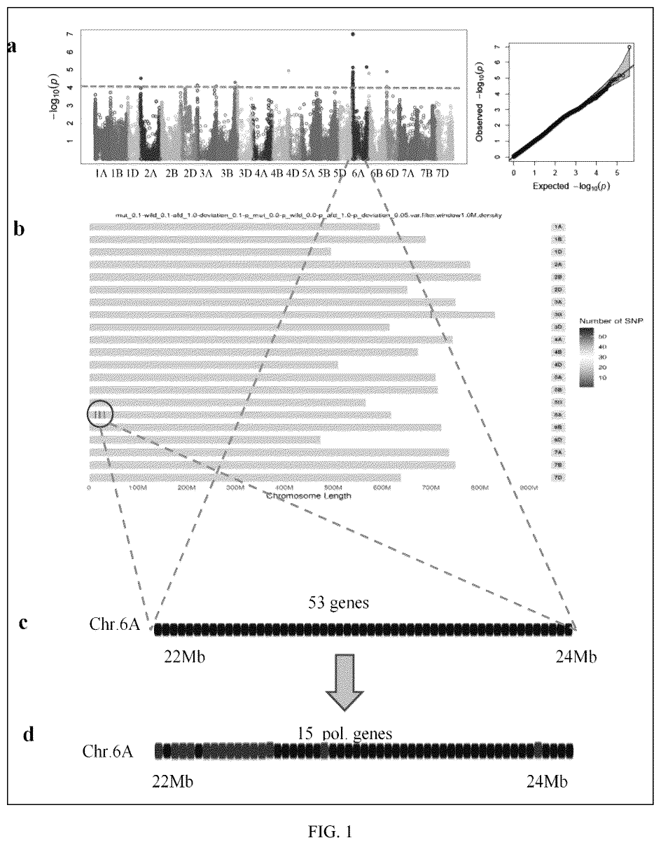

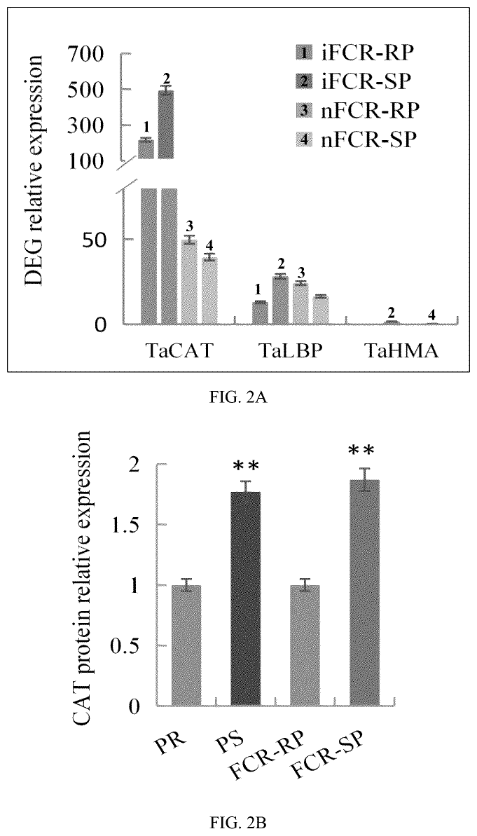

A millet medium inoculation method is used to identify resistance of the natural population consisting of 243 wheat varieties in the Huanghuai wheat region to the FCR, and GWAS is performed (part a in FIG. 1). The resistant and susceptible materials of the two F₄ generation populations (JY, JZ) are used for WES (part b in FIG. 1). A resistance segment is co-localized in 22 mega base pairs (Mb) to 24 Mb segment of a chromosome 6A of the wheat, which contains 53 annotated genes, of which the promoters or coding regions of 15 genes are different in the resistant and susceptible materials (FIG. 1). Based on the bulking transcriptome sequencing analysis of the JY population, 3 out of the 15 genes can be induced by the FCR, that is, TaCAT (TraesCS6A02G041700), TaLBP (TraesCS6A02G042600) and TaHMA (TraesCS6A02G043000). Specifically, an expression level of the TaCAT gene in each pool is significantly higher than that of TaLBP and TaHMA (FIG. 2A). Based on the bulking proteome sequencing analysis of the JY population, protein coded by the TaCAT gene can be detected in the above co-located segments, and difference of the expression level of the TaCAT gene between the parent pool and the descendant pool is significant (FIG. 2B). In summary, the TaCAT gene is related to the FCR of wheat.

A Primer 5 software is used to design primers to amplify the TaCAT gene. A forward primer of the primer pair is an upstream primer with the nucleotide sequence as shown in SEQ ID NO: 5, and a reverse primer of the primer pair is a downstream primer with the nucleotide sequence as shown in SEQ ID NO: 6.

The nucleotide sequences of the upstream primer and the downstream are as follows:

TaCAT-F1 as shown in SEQ ID NO: 5:
5′-AGAGAGCAGTGCAGTGAGAG-3′;
and
TaCAT-R1 as shown in SEQ ID NO: 6:
5′-GGCTAGCAGACCGAAAACAG-3′.

The above primer pair is used to amplify the genome of the TaCAT gene of wheat materials with obvious resistance and sensitivity separation, such as JM1, YN805 and ZM082, the PCR amplification system is shown in Table 2, and the PCR amplification program is shown in Table 3.

TABLE 2
PCR amplification system of the primer TaCAT-F1
and the primer TaCAT-R1 to the TaCAT gene

| Component | Volume | |
|---|---|---|
| 2 × Phanta Max buffer | 25 | microliters (μL) |
| dNTP Mix(10 millimolar per liter abbreviated as mM each) | 1 | μL |
| Template cDNA | 2 | μL |
| Primers F/R (i.e., the forward primer and the reverse primer, 10 | 2/2 | μL |
| micromolar per liter abbreviated as μM) | | |
| Phanta Max Super-Fidelity DNA Polymerase | 1 | μL |
| Double distilled water (dd H₂O) | 17 | μL |

TABLE 3
PCR amplification program of the primer TaCAT-F1
and the primer TaCAT-R1 to the TaCAT gene

| — | Process | Temperature | Time | |
|---|---|---|---|---|
| Step 1 | Pre-denaturation | 95 Celsius degree | 5 | minutes (min) |
| | | (° C.) | | |
| Step 2 | Denaturation | 95° C. | 30 | seconds (s) |
| 34 cycles | Annealing | Melting temperature | 30 | s |
| | | (Tm) | | |
| | Extension | 72° C. | 2 | min |
| Step 3 | Thorough extension | 72° C. | 10 | min |

| | | | |
|---|---|---|---|
| Step 4 | Stored at 4° C. | — | — |

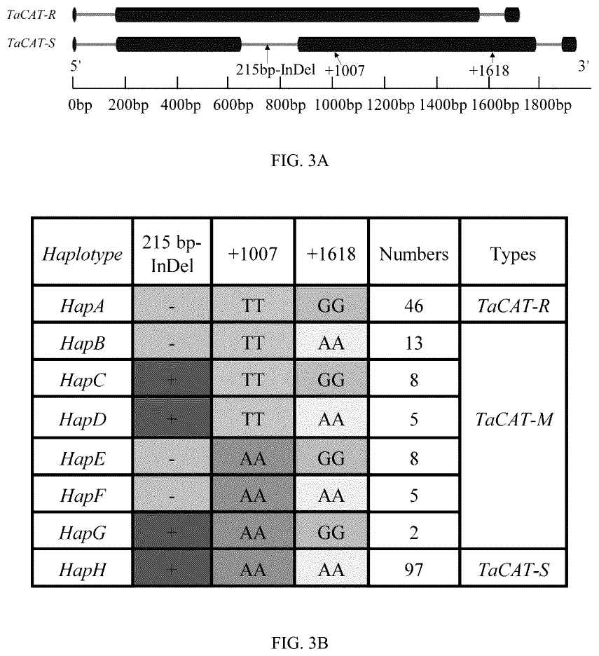

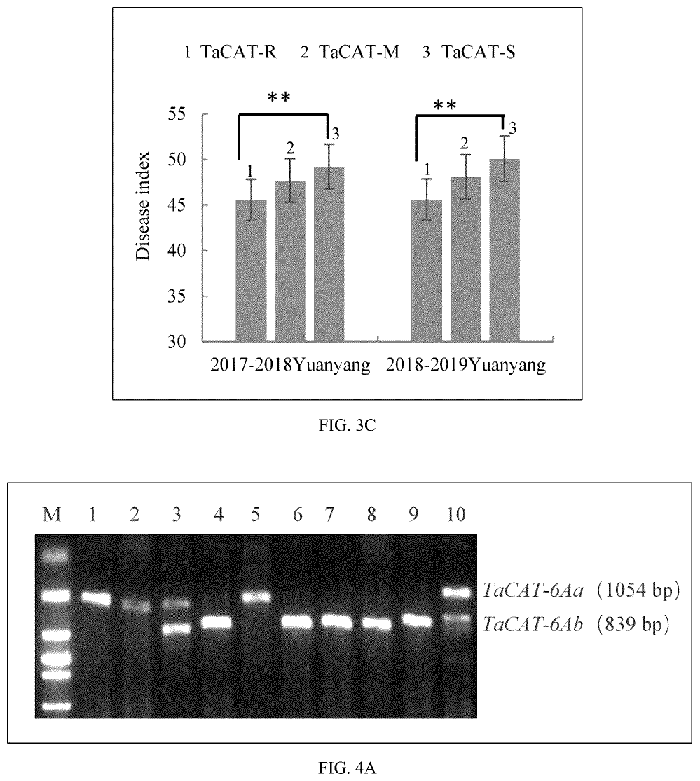

It is discovered through sequencing analysis that the TaCAT gene of the susceptible material has an additional intron with 215 bp than that of the resistant material. Meanwhile, there are two amino acid site differences in the gene coding region, namely Thr214Ser and Arg418Lys (FIG. 3A). The sequencing analysis is further performed on the 243 wheat materials in the natural population to discover that the difference sites of the TaCAT gene in the resistant and susceptible materials are clearly separated, which forms a total of 8 haplotypes and 3 genotypes (FIG. 3B). Based on the phenotyping, it is found that the resistance differences among the three genotypes are significant (FIG. 3C), which indicates that the TaCAT gene is significantly related to the FCR of wheat.

### Embodiment 2 Development and Application of an Allele Marker of the TaCAT Gene

According to the nucleotide sequence of the TaCAT gene as shown in SEQ ID NO: 1, there is an insertion or deletion of 215 bp between the resistant and susceptible materials staring from the 654^{th} position after a start codon ATG. An InDel marker is developed according to the insertion or deletion fragment to detect different genotypes of the TaCAT gene. The differential site-specific primer pair includes: an upstream primer with the nucleotide sequence as shown in SEQ ID NO: 7:5′-CCCGATCTTGCTGGAGGA-3′, and a downstream primer with the nucleotide sequence as shown in SEQ ID NO: 8: 5′-AAGACGCGCCAATACTCCTG-3′.

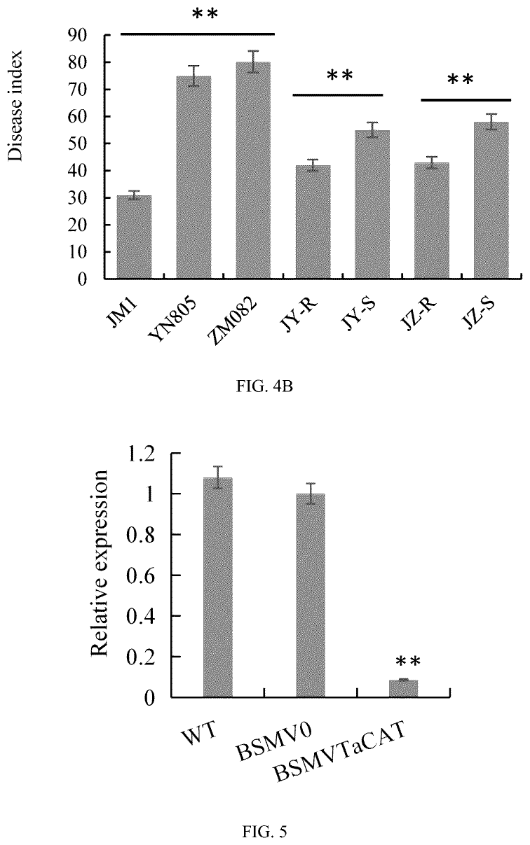

The specific InDel marker is used to amplify DNA from different materials. A genotype of a wheat variety/strain with an amplification product of 1054 bp is named as TaCAT-6Aa, a genotype of a wheat variety/strain with an amplification product of 839 bp is named as TaCAT-6Ab (FIG. 4A), and there is a significant difference in the disease resistance ability of plants corresponding to the two genotypes.

The developed InDel marker of the TaCAT gene is further detected and verified in the JY and JZ populations to obtain results as shown in FIG. 4B, that is, the two alleles of TaCAT-6Aa and TaCAT-6Ab in the populations are clearly separated, and the corresponding resistance to the FCR of wheat is significantly different. This marker can be used as a detection marker and an auxiliary selection marker for disease-resistance for the TaCAT gene of the wheat.

### Embodiment 3 VIGS Silencing and Phenotypic Identification of the TaCAT Gene

### (1) Construction of a VIGS Vector of the TaCAT Gene

Primers are designed according to the cDNA sequence of the TaCAT gene, and a restriction enzyme cutting site and a protective base are added to the 5′ ends of the forward and reverse primers, respectively, to form the upstream primer TaCAT-F2 as shown in SEQ ID NO: 9: 5′-AGCTAGCTGATTAATTAAGCAACAACTTCCCCGTCTTC-3′, and the downstream primer TaCAT-R2 as shown in SEQ ID NO: 10: 5′-TGCTAGCTGAGCGGCCGCAAGAAGAAGGTGTGGAGGCT-3′. The VIGS fragment of the TaCAT gene is amplified by using the primer pair and recovered for later use. The γ-PDS vector is double-digested with PacI and NotI to obtain a γ-PDS linearized vector fragment. The recovered fragment and the γ-PDS linearized vector fragment are used to complete the construction of the recombinant vector (γ-TaCAT) by a seamless cloning method.

### (2) Plant VIGS Experiment of the TaCAT Gene

The viral vector (α, β, γ, γ-PDS) and the TaCAT gene silencing recombinant vector (γ-TaCAT) plasmids are linearized by digestion, and the linearized plasmids are further transcribed in vitro by using a RiboMAX™ Large Scale RNA Production Systems-T7 kit to obtain different components of viral in vitro transcription products. 2.5 μL of each of the in vitro transcription products α, β, γ/γ-PDS/γ-TaCAT is taken, mixed with a ratio of 1:1:1, and diluted with an equal volume of diethyl pyrocarbonate (DEPC) water to obtain diluted mixture. 5 μL of the diluted mixture is taken and added to 90 μL of FES buffer solution to obtain a mixture, and the mixture is mixed thoroughly by pipetting to obtain a FES mixed solution. Four different treatments are set up for each experiment, including: a complete blank control group (WT), a virus blank control group (α+β+γ), an albino positive control group (α+β+γ-PDS) and a gene silencing group (α+β+γ-TaCAT).

Specifically, the FES buffer solution is obtained by the following steps: glycine is added with dipotassium hydrogenphosphate to obtain a mother liquid, and sodium pyrophosphate, bentonite and diatomaceous earth are added into the mother liquid to obtain the FES buffer solution.

When the virus is infected, a small amount of the DEPC water is sprayed on the surface of the plant to be infected (JM1). 10 μL of the FES mixed solution is applied on a clean glove, the second leaf of the seedling is rubbed from the base to the tip for three times with controlled strength by the glove, and a little DEPC water is sprayed from top to bottom to maintain humidity after rubbing. The glove need to be changed to a clean one for each treatment. After the virus is inoculated, it is placed in a 23+2° C. incubator to keep warm, moisturize and avoid light for 24 h, and then adjust it to a 16 h/8 h light and dark cycle for cultivation, and observe and record phenotypic changes regularly.

### (3) Phenotypic Identification after VIGS for the TaCAT Gene

After 2 weeks of VIGS for the TaCAT gene, the relative expression levels of the TaCAT gene in a silenced plant (BSMVTaCAT), a virus-free plant (BSMV0) and a non-silenced plant (WT) are detected by a qRT-PCR technology. The result shows that the relative expression level of the TaCAT gene in the silenced plant is significantly downregulated, and the gene is effectively silenced (FIG. 5).

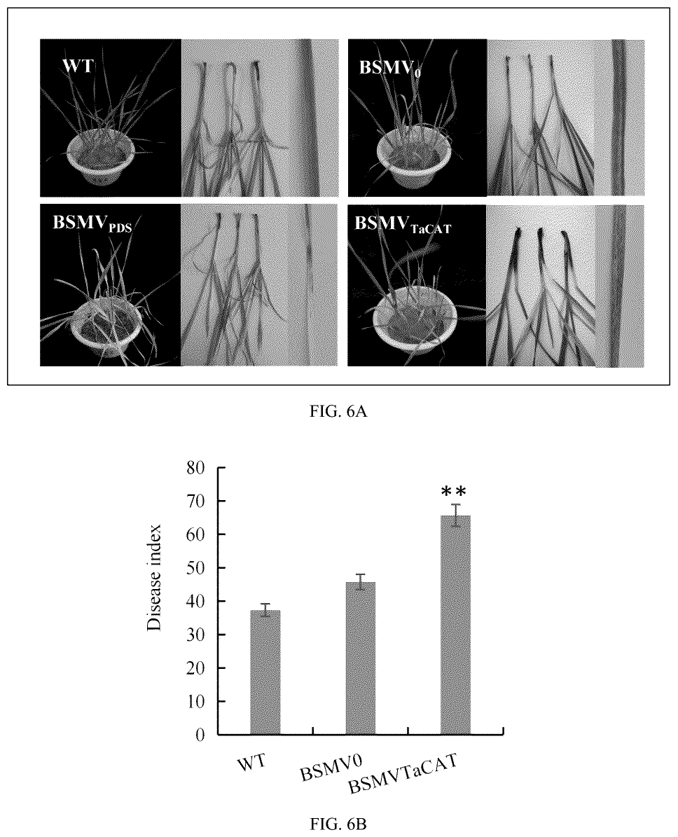

The phenotype of the silenced plant for the FCR is further identified, and the result shows that the disease index of the TaCAT gene-silenced plant (disease index abbreviated as DI=67.90) is significantly higher than that of the virus-free plant (JM1, DI=37.66), and the disease-resistance of the plants is significantly reduced (FIG. 6A and 6B).

In summary, the disease-resistance of plants with silenced TaCAT gene is significantly reduced, which indicates that the TaCAT gene is a gene that positively regulates resistance to the FCR of wheat.

### Embodiment 4 Verification of an Ethyl Methane Sulfonate (EMS) Mutant of the TaCAT Gene

The disease-resistance of the TaCAT gene is further verified by using a tetraploid mutant induced by EMS. Kronos3868 is a premature termination mutant of the TaCAT gene, and the nucleotide at 396^{th} bp after the 5′ end ATG of the TaCAT gene sequence mutates from G to A, causing the gene to terminate prematurely. After backcrossing the Kronos3868 positive mutant with the wild-type Kronos-WT twice to eliminate the background difference of the materials, the resistance of the backcrossed Kronos3868 (named cat1) and the wild-type Kronos-WT materials to the FCR of wheat is further identified at the seedling and adult plant stages.

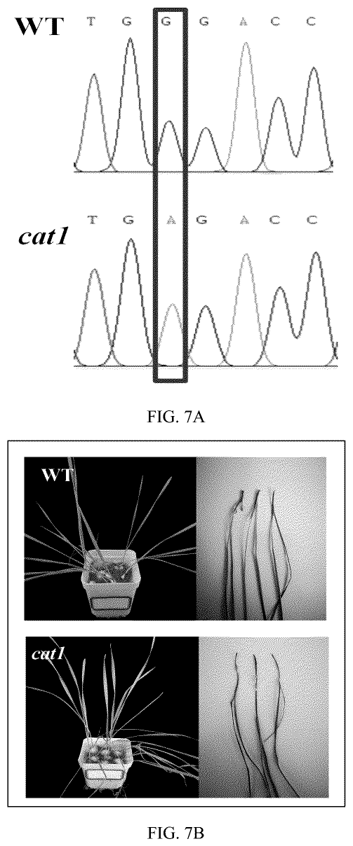

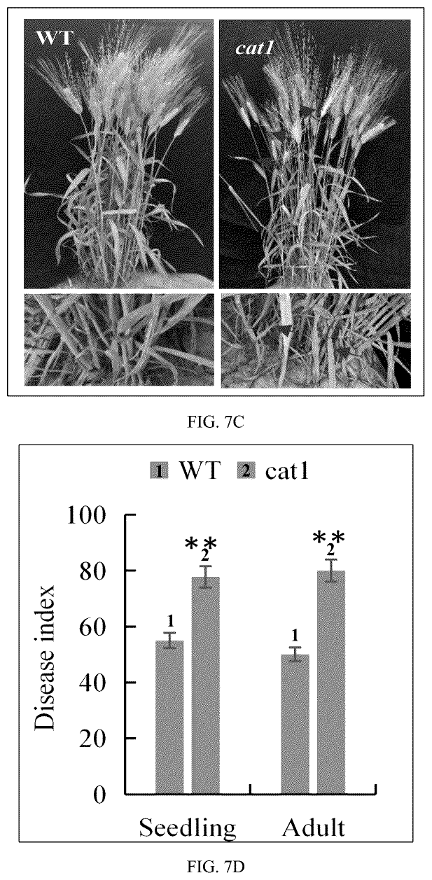

The result shows that the disease indexes corresponding to cat1 (DI_{seedling stage}=77.78, DI_{adult plant stage}=80) are significantly higher than that of Kronos-WT (DI_{seedling stage}=55, DI_{adult plant stage}=50) whether the plant is in the seedling stage or the adult plant stage, and the disease-resistance of the plants is significantly reduced (FIGS. 7A-7D). It is speculated that the TaCAT gene plays a positive role in regulating the resistance to FCR of wheat in the plant.

### Embodiment 5 Acquisition and Identification of a TaCAT Transgenic Wheat

### (1) Construction of a TaCAT Plant Overexpression Vector

Primers are designed based on the full-length cDNA sequence of the TaCAT gene in Chinese spring (also refers to Triticum aestivum, with a genome sequence of IWGSC RefSeq v2.1), and recognition sites and protective bases of restriction endonucleases BamHI and SacI are introduced at both ends of the primers. The primer sequences are as follows:
TaCAT-F3 as shown in SEQ ID NO: 11: 5′-GATGACGATGACAAGGGATCCGATCCCTGCAAGTTCCGGC-3′ (bases with the underlined are the recognition sites and protection bases of the restriction endonuclease BamHI); and
TaCAT-R3 as shown in SEQ ID NO: 12: 5′-ACGAACGAAAGCTCTGAGCTCTCACATGCTTGGCTTCACG-3′ (bases with the underlined are the recognition sites and protection bases of the restriction endonuclease SacI).

A TRIzol reagent is used to extract total RNA from the seedling stage of JM1. The RNA is used as a template, and a SuperScript III reverse transcriptase (Invitrogen, Cat NO: 18080-044) is used for reverse transcription to obtain cDNA. The cDNA is used as a template, and the PCR amplification is performed on a coding sequence of the TaCAT gene of the wheat by using the primers TaCAT-F3 and TaCAT-R3. The amplification product is recovered and purified for later use.

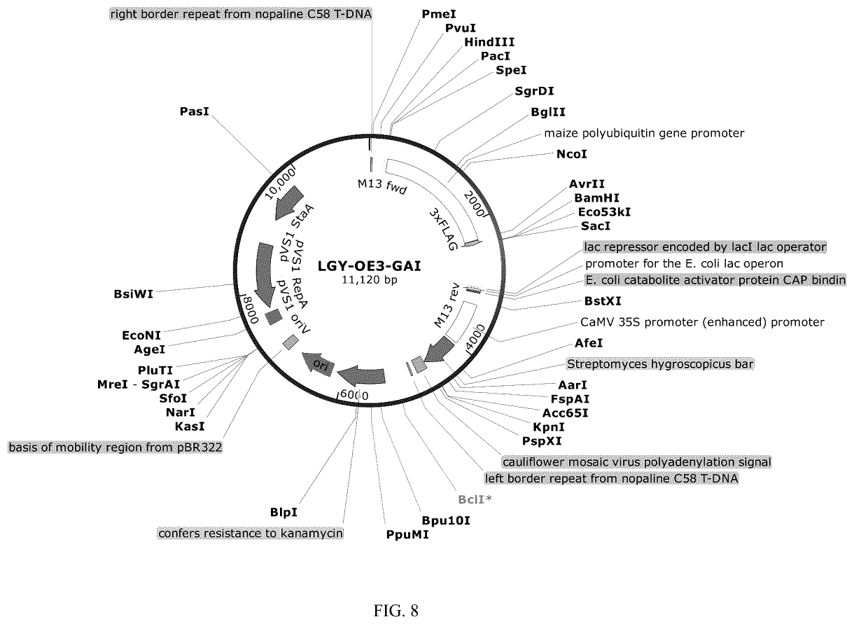

The plant expression vector pLGY-OE3 is selected, and its vector map is shown in FIG. 8.

The plant expression vector is double-digested with BamHI and SacI, recovered and purified to obtain a purified plant expression vector, and the recombinant vector (pLGY-OE3-TaCAT) is constructed by the purified plant expression vector with the above DNA fragment.

### (2) Acquisition of the TaCAT Transgenic Wheat

Immature embryo callus of wheat KN199 is transformed by using the pLGY-OE3-TaCAT and an Agrobacterium infection method, and positive plants are obtained after screening, pre-differentiation and differentiation. The PCR amplification is performed on the positive plants by using primers TaCAT-F4 and TaCAT-R4 to obtain amplification products, and the amplification products are sequenced to obtain T₀ generation transgenic plants.

The sequences of the primers include:

TaCAT-F4 as shown in SEQ ID NO: 13:
5′-ATTCATATGCTCTAACCTTGAGT-3′;
and
TaCAT-R4 as shown in SEQ ID NO: 14:
5′-TGGGCGATCTTCTCCAGCAG-3′.

The PCR amplification system is shown in Table 4, and the PCR amplification program is shown in Table 5.

TABLE 4
PCR amplification system of the primers
TaCAT-F4 and TaCAT-R4 for the TaCAT gene

| Component | Volume | |
|---|---|---|
| 5 × Mix Enzyme | 5 | μL |
| TaCAT-F4 | 0.5 | μL |
| TaCAT-R4 | 0.5 | μL |
| DNA | 1 | μL |

| | |
|---|---|
| dd H₂O | To 10 μL |

TABLE 5
PCR amplification program of the primers
TaCAT-F4 and TaCAT-R4 for the TaCAT gene

| — | Process | Temperature | Time | |
|---|---|---|---|---|
| Step 1 | Pre-denaturation | 95° C. | 5 | min |
| Step 2 | Denaturation | 95° C. | 30 | s |
| 34 cycles | Annealing | Tm | 30 | s |
| | Extension | 72° C. | 40 | s |
| Step 3 | Thorough extension | 72° C. | 10 | min |

| | | | |
|---|---|---|---|
| Step 4 | Stored at 4° C. | — | — |

### (3) PCR Identification and Phenotypic Identification of the Transgenic Wheat

The PCR amplification is performed on overexpression plants by using the primers TaCAT-F4 and TaCAT-R4 to obtain amplification products, and the amplification products are sequenced to screen positive transgenic plants, and a total of 10 positive transgenic lines are obtained. After additional generations, a T₂ generation transgenic line is obtained.

The primers TaCAT-F5 and TaCAT-R5 are used for fluorescence real-time quantitative PCR for detecting the expression level of the TaCAT gene in the leaves of the T₂ generation positive transgenic plants.

The sequences of the primers include:

TaCAT-F5 as shown in SEQ ID NO: 15:
5′-ACACCTACACCTTCGTCTCC-3;
and
TaCAT-R5 as shown in SEQ ID NO: 16:
5′-GCACGAACAGCTTCCACTC-3′.

The PCR amplification system is shown in Table 6, and the PCR amplification program is shown in Table 7.

TABLE 4
PCR amplification system of the primers
TaCAT-F5 and TaCAT-R5 for the TaCAT gene

| Component | Volume | |
|---|---|---|
| 2 × SYBR Premix | 5 | μL |
| Forward Primer (TaCAT-F5) | 0.5 | μL |
| Reverse Primer (TaCAT-R5) | 0.5 | μL |
| cDNA | 2 | μL |
| Nuclease-Free Water | 2 | μL |

TABLE 7
PCR amplification program of the primers
TaCAT-F5 and TaCAT-R5 for the TaCAT gene

| Temperature | Time | Cycles |
|---|---|---|
| 95° C. | 30 s | 1 |
| 95° C. | 10 s | 45 |
| 60° C. | 20 s | 45 |
| 72° C. | 20 s | 45 |

The result is shown in FIG. 9. The expression level of the target TaCAT gene in the leaves of the T₂ generation positive transgenic plants is significantly higher than that of the wild-type KN199, and is about 18 to 22 times the expression level in the empty leaves.

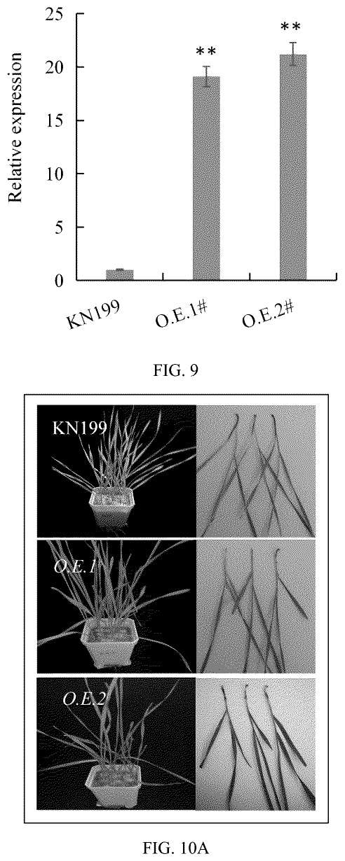

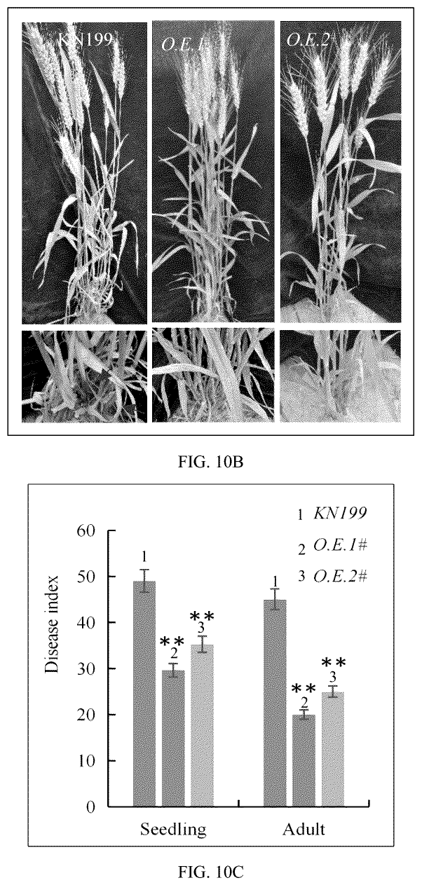

The overexpression transgenic lines T₂ (TaCAT-OX1 (i.e. O.E.1 #), TaCAT-OX2 (i.e. O.E.2 #)) and wild-type KN199 are further identified for resistance to the FCR at the seedling and adult plant stages. The identification results are shown in FIG. 10.

Compared with the wild-type KN199 plants, the disease indexes of the overexpressing transgenic lines T₂ (TaCAT-OX1 andTaCAT-OX2) to the FCR is significantly reduced, and the disease-resistance of the plants is significantly improved (FIGS. 10A-10C).

In summary, the TaCAT gene related to the FCR of wheat of the disclosure can regulate resistance to the FCR of wheat, can be further used to study the molecular mechanism related to controlling the FCR of wheat, and has important application value in wheat disease-resistance breeding.

The embodiments described above are merely some of the embodiments of the disclosure, and are not intended to limit the scope of implementation of the disclosure. Therefore, any equivalent changes or amendments made according to the structures, features and principles described in the patent scope of the disclosure should be included in the scope of the disclosure.

What is claimed is:

1. An application method of a TaCAT gene as shown in SEQ ID NO: 1, a complementary deoxyribonucleic acid (cDNA) of the TaCAT gene as shown in SEQ ID NO: 2, a coding sequence (CDS) of the TaCAT gene as shown in SEQ ID NO: 3, and a protein coded by the TaCAT gene as shown in SEQ ID NO: 4, comprising:
breeding a wheat variety resistant to Fusarium crown rot by using the TaCAT gene, the cDNA of the TaCAT gene, the CDS of the TaCAT gene and the protein coded by the TaCAT gene.

2. The application method as claimed in claim 1, wherein the application method specifically comprises:
transforming the TaCAT gene as shown in SEQ ID NO: 1, the cDNA of the TaCAT gene as shown in SEQ ID NO: 2, or the CDS of the TaCAT gene as shown in SEQ ID NO: 3 into a target plant to obtain a transgenic plant for adjusting resistance to the Fusarium crown rot.

3. The application method as claimed in claim 1, wherein the application method specifically comprise:
expressing the protein coded by the TaCAT gene as shown in SEQ ID NO: 4 in a target plant to obtain a transgenic plant for adjusting resistance to the Fusarium crown rot.

4. A product for detecting an allelotype of Fusarium crown rot of wheat, comprising:
(1) a primer combination for detecting the allelotype of the Fusarium crown rot of wheat, wherein the primer combination comprises:
an upstream primer with the nucleotide sequence as shown in SEQ ID NO: 5, and a downstream primer with the nucleotide sequence as shown in SEQ ID NO: 6; or
an upstream primer with the nucleotide sequence as shown in SEQ ID NO: 7, and a downstream primer with the nucleotide sequence as shown in SEQ ID NO: 8;
(2) a reagent or a kit comprising the primer combination.

5. An application method of the product as claimed in claim 4, comprising at least one of:
detecting the allelotype of the Fusarium crown rot of wheat by using the product;
performing molecular marker assisted breeding of wheat by using the product;
constructing a genetic map of wheat by using the product.

6. A method for detecting an allelotype of Fusarium crown rot of wheat, comprising:
step 1, extracting genomic DNA of a to-be-detected wheat;
step 2, taking the genomic DNA of the to-be-detected wheat as a template, performing a polymerase chain reaction (PCR) amplification with an upstream primer and a downstream primer to obtain an amplification product, and detecting the allelotype of the Fusarium crown rot of wheat.

7. The method for detecting the allelotype of the Fusarium crown rot of the wheat as claimed in claim 6, wherein the performing a PCR amplification with an upstream primer and a downstream primer to obtain an amplification product, and detecting the allelotype of the Fusarium crown rot of wheat, comprises:
performing the PCR amplification with an upstream primer with the nucleotide sequence as shown in SEQ ID NO: 5, and a downstream primer with the nucleotide sequence as shown in SEQ ID NO: 6, to obtain the amplification product as shown in SEQ ID NO: 1, and detecting the allelotype of the Fusarium crown rot of wheat based on the amplification product as shown in SEQ ID NO: 1; and
wherein a resistant and susceptible material has an insertion or deletion (InDel) of 215 base pairs (bp) starting from a 654^{th} position after a start codon adenine-thymine-guanine (ATG) in the gene sequence as shown in SEQ ID NO: 1, and a genotype of the amplification product is determined according to the InDel; and a genotype corresponding to an insertion type amplification product is named as TaCAT-6Aa, and a genotype corresponding to a deletion type amplification product is named as TaCAT-6Ab; and
wherein resistance of a plant with the genotype of TaCAT-6Aa to the Fusarium crown rot of wheat is lower than resistance of a plant with the genotype of TaCAT-6Ab to the Fusarium crown rot of wheat.

8. The method for detecting the allelotype of the Fusarium crown rot of wheat as claimed in claim 6, wherein the performing a PCR amplification with an upstream primer and a downstream primer to obtain an amplification product, and detecting the allelotype of the Fusarium crown rot of wheat, comprises:
performing the PCR amplification with an upstream primer with the nucleotide sequence as shown in SEQ ID NO: 7, and a downstream primer with the nucleotide sequence as shown in SEQ ID NO: 8, to obtain the amplification product, and detecting the allelotype of the Fusarium crown rot of wheat based on the amplification product; wherein a genotype with the amplification product of 1054 bp is TaCAT-6Aa, and a genotype with the amplification product of 839 bp is TaCAT-6Ab;
wherein resistance of a plant with the genotype of TaCAT-6Aa to the Fusarium crown rot of wheat is lower than resistance of a plant with the genotype of TaCAT-6Ab to the Fusarium crown rot of wheat.

9. An application method of the product as claimed in claim 4, comprising:
breeding a wheat variety resistant to the Fusarium crown rot by using the product.


SEQUENCE LISTING (16 sequences)

SEQ ID NO: 1 - DNA, 2470 bp, Triticum aestivum
  source 1..2470: mol_type = other DNA
aagaccggctcgagcgagctcaggcagagcagagagcagtgcagtgagagtgagtgagctcgccacactccccccagcttagcggagctacctcactcaagagaggaaggatggatccctgcaaggtaagcagacacgatcagactaatcctatgctactttacagtccatggtgttcttaattcgttcacatagattctaccgattatagtacgtgtgtgcgtgtgctgattaattaacatcgagctctgtgtgcgtgcgtgtgcatggcgtgcagttccggccgtcgagcagcttcgacaccaagacgacgacgaccaacgccgggcagccggtgtggaacgacaacgaggcgctcaccgtcggcccgcgcggcccgatcttgctggaggactaccacctgctggagaagatcgcccacttcgcgcgcgagcgcatcccggagcgggtggtgcacgcgcggggcgcctccgccaagggcttcttcgagtgcacccacgacgtgacgggcttgacctgcgccgacttcctccgcgcgccgggggcgcgcacgccggtcatcgtccgcttttccacggtgatccacgagcgcggctcgccggagacgatccgtgacccgcgcgggttcgccgtcaagttctacacgcgcgagggcaactgggacctgctcggcaacaacttccccgtcttcttcatccgcgacggcatcaagttccccgacgtcatccatgccttcaagcccaacccaaagtcccacgtgcaggcaaggttttatttatttatttcggaaacaagataatttctagcctcatccaaatatgcaaattttcgaaaatgaattctagcataacttaaatttattttaaaaccagttaatttgaatcttccgtgaaatttcagggttaaaaatgttcctgacctcactgaaatatactaaattttggaaatttctttggatttttttaaccttgtgtgcaggagtattggcgcgtcttcgacttcctttcccaccacccggagagcctccacaccttcttcttcctcttcgacgacgtcggcatcccgaccgactaccgccacatggacggcttcggcgtcaacacctacaccttcgtcacccgcgccggcaagtcccactacgtcaagttccactggaggcccacctgcggcgtcagctgcctcatggacgacgaggccacgctcgtcggcggcaagaaccacagccacgccacccaggacctctacgactccatcgacgccggcaacttccccgagtggaagctgttcgtgcaggtcatcgaccccgacgacgaggaccgcttcgacttcgacccgctcgacgacaccaagacatggcccgaggacctcgtccccctccagcccgtcggccgcctcgtgctcgaccgcaacgtcgacaacttcttcaacgagaacgagcagctggccttcggcccggggctcgtcgtgcctgggatctactactccgacgacaagatgctgcagtgcagggtgttcgcctacgccgacacgcagcgctaccgcctcggcccaaactacctgatgctccccgtcaacgcgcccaagtgcggcttcaagaacaaccactacgacggcgccatgaacttcatgcaccgcgacgaggaggtggactactacccgtcccgccacgcgccgctccggcaggcggagccggccagcttccctgtgcccacgcggcccgtcgtcggaagaagggagaagacgaggatcaagaaggagaacgacttcgtgcagcccggggagaggtaccgcagctgggcgcccgaccgccaggacaggttcgtgcgccgcttcgccgacgccctcgcccaccccaaggtcagccacgagctccgcgtcatctggatcgacttcctctccaaggtaactaattgttgatgcatgaacccatggatgtacgctcaatgttcttcagttggtgatgaatgattaactcggttgatttgtggatggatgcagtgtgacaagtcgtgcggcatgaaggtggcgaaccgtctcaacgtgaagccaagcatgtgatggatggaccgttctgtgtaaactttgtgctatatcatggaccagcaataagagaccatgccagctataatgtggatcatgtggtcgattacctactgtgctgctactaataagttactatcattttcttttatgtcgcagtaacttacctgtaatccagcaaatgaaattaaagtgggtgctttggagcttatgaatccaaacgtgtattcgtctttgcctcagatgatatttgctgttctggatggaacaagatggctctgttttcggtctgctagccacatcaaagtttgttttgcacgaccttctgtgacaactttgagtaacaaagagcggaaattagaaagtgcacccgctccctgcccccaaagaacaattccccaatgtaaaaagatactacatcgatccgtct

SEQ ID NO: 2 - DNA, 1845 bp, Triticum aestivum
  source 1..1845: mol_type = genomic DNA
agagagcagtgcagtgagagtgagtgagctcgccacactccccccagcttagcggagctacctcactcaagagaggaaggatggatccctgcaagttccggccgtcgagcagcttcgacaccaagacgacgacgaccaacgccgggcagccggtgtggaacgacaacgaggcgctcaccgtcggcccgcgcggcccgatcttgctggaggactaccacctgctggagaagatcgcccacttcgcgcgcgagcgcatcccggagcgggtggtgcacgcgcggggcgcctccgccaagggcttcttcgagtgcacccacgacgtgacgggcttgacctgcgccgacttcctccgcgcgccgggggcgcgcacgccggtcatcgtccgcttttccacggtgatccacgagcgcggctcgccggagacgatccgtgacccgcgcgggttcgccgtcaagttctacacgcgcgagggcaactgggacctgctcggcaacaacttccccgtcttcttcatccgcgacggcatcaagttccccgacgtcatccatgccttcaagcccaacccaaagtcccacgtgcaggagtattggcgcgtcttcgacttcctttcccaccacccggagagcctccacaccttcttcttcctcttcgacgacgtcggcatcccgaccgactaccgccacatggacggcttcggcgtcaacacctacaccttcgtcacccgcgccggcaagtcccactacgtcaagttccactggaggcccacctgcggcgtcagctgcctcatggacgacgaggccacgctcgtcggcggcaagaaccacagccacgccacccaggacctctacgactccatcgacgccggcaacttccccgagtggaagctgttcgtgcaggtcatcgaccccgacgacgaggaccgcttcgacttcgacccgctcgacgacaccaagacatggcccgaggacctcgtccccctccagcccgtcggccgcctcgtgctcgaccgcaacgtcgacaacttcttcaacgagaacgagcagctggccttcggcccggggctcgtcgtgcctgggatctactactccgacgacaagatgctgcagtgcagggtgttcgcctacgccgacacgcagcgctaccgcctcggcccaaactacctgatgctccccgtcaacgcgcccaagtgcggcttcaagaacaaccactacgacggcgccatgaacttcatgcaccgcgacgaggaggtggactactacccgtcccgccacgcgccgctccggcaggcggagccggccagcttccctgtgcccacgcggcccgtcgtcggaagaagggagaagacgaggatcaagaaggagaacgacttcgtgcagcccggggagaggtaccgcagctgggcgcccgaccgccaggacaggttcgtgcgccgcttcgccgacgccctcgcccaccccaaggtcagccacgagctccgcgtcatctggatcgacttcctctccaagtgtgacaagtcgtgcggcatgaaggtggcgaaccgtctcaacgtgaagccaagcatgtgatggatggaccgttctgtgtaaactttgtgctatatcatggaccagcaataagagaccatgccagctataatgtggatcatgtggtcgattacctactgtgctgctactaataagttactatcattttcttttatgtcgcagtaacttacctgtaatccagcaaatgaaattaaagtgggtgctttggagcttatgaatccaaacgtgtattcgtctttgcctcagatgatatttgctgttctggatggaacaagatggctctgttttcggtctgctagcc

SEQ ID NO: 3 - DNA, 1485 bp, Triticum aestivum
  source 1..1485: mol_type = genomic DNA
atggatccctgcaagttccggccgtcgagcagcttcgacaccaagacgacgacgaccaacgccgggcagccggtgtggaacgacaacgaggcgctcaccgtcggcccgcgcggcccgatcttgctggaggactaccacctgctggagaagatcgcccacttcgcgcgcgagcgcatcccggagcgggtggtgcacgcgcggggcgcctccgccaagggcttcttcgagtgcacccacgacgtgacgggcttgacctgcgccgacttcctccgcgcgccgggggcgcgcacgccggtcatcgtccgcttttccacggtgatccacgagcgcggctcgccggagacgatccgtgacccgcgcgggttcgccgtcaagttctacacgcgcgagggcaactgggacctgctcggcaacaacttccccgtcttcttcatccgcgacggcatcaagttccccgacgtcatccatgccttcaagcccaacccaaagtcccacgtgcaggagtattggcgcgtcttcgacttcctttcccaccacccggagagcctccacaccttcttcttcctcttcgacgacgtcggcatcccgaccgactaccgccacatggacggcttcggcgtcaacacctacaccttcgtcacccgcgccggcaagtcccactacgtcaagttccactggaggcccacctgcggcgtcagctgcctcatggacgacgaggccacgctcgtcggcggcaagaaccacagccacgccacccaggacctctacgactccatcgacgccggcaacttccccgagtggaagctgttcgtgcaggtcatcgaccccgacgacgaggaccgcttcgacttcgacccgctcgacgacaccaagacatggcccgaggacctcgtccccctccagcccgtcggccgcctcgtgctcgaccgcaacgtcgacaacttcttcaacgagaacgagcagctggccttcggcccggggctcgtcgtgcctgggatctactactccgacgacaagatgctgcagtgcagggtgttcgcctacgccgacacgcagcgctaccgcctcggcccaaactacctgatgctccccgtcaacgcgcccaagtgcggcttcaagaacaaccactacgacggcgccatgaacttcatgcaccgcgacgaggaggtggactactacccgtcccgccacgcgccgctccggcaggcggagccggccagcttccctgtgcccacgcggcccgtcgtcggaagaagggagaagacgaggatcaagaaggagaacgacttcgtgcagcccggggagaggtaccgcagctgggcgcccgaccgccaggacaggttcgtgcgccgcttcgccgacgccctcgcccaccccaaggtcagccacgagctccgcgtcatctggatcgacttcctctccaagtgtgacaagtcgtgcggcatgaaggtggcgaaccgtctcaacgtgaagccaagcatgtga

SEQ ID NO: 4 - protein, 494 aa, Triticum aestivum
MDPCKFRPSSSFDTKTTTTNAGQPVWNDNEALTVGPRGPILLEDYHLLEKIAHFARERIPERVVHARGASAKGFFECTHDVTGLTCADFLRAPGARTPVIVRFSTVIHERGSPETIRDPRGFAVKFYTREGNWDLLGNNFPVFFIRDGIKFPDVIHAFKPNPKSHVQEYWRVFDFLSHHPESLHTFFFLFDDVGIPTDYRHMDGFGVNTYTFVSRAGKSHYVKFHWRPTCGVSCLMDDEATLVGGKNHSHATQDLYDSIDAGNFPEWKLFVQVIDPDDEDRFDFDPLDDTKTWPEDLVPLQPVGRLVLDRNVDNFFNENEQLAFGPGLVVPGIYYSDDKMLQCRVFAYADTQRYRLGPNYLMLPVNAPKCGFKNNHYDGAMNFMHRDEEVDYYPSRHAPLRQAEPASFPVPTRPVVGKREKTRIKKENDFVQPGERYRSWAPDRQDRFVRRFADALAHPKVSHELRVIWIDFLSKCDKSCGMKVANRLNVKPSM

SEQ ID NO: 5 - DNA, 20 bp, Triticum aestivum
  source 1..20: mol_type = other DNA
agagagcagtgcagtgagag

SEQ ID NO: 6 - DNA, 20 bp, synthetic construct
  source 1..20: mol_type = other DNA
ggctagcagaccgaaaacag

SEQ ID NO: 7 - DNA, 18 bp, synthetic construct
  source 1..18: mol_type = other DNA
cccgatcttgctggagga

SEQ ID NO: 8 - DNA, 20 bp, synthetic construct
  source 1..20: mol_type = other DNA
aagacgcgccaatactcctg

SEQ ID NO: 9 - DNA, 38 bp, synthetic construct
  source 1..38: mol_type = other DNA
agctagctgattaattaagcaacaacttccccgtcttc

SEQ ID NO: 10 - DNA, 38 bp, synthetic construct
  source 1..38: mol_type = other DNA
tgctagctgagcggccgcaagaagaaggtgtggaggct

SEQ ID NO: 11 - DNA, 40 bp, synthetic construct
  source 1..40: mol_type = other DNA
gatgacgatgacaagggatccgatccctgcaagttccggc

SEQ ID NO: 12 - DNA, 40 bp, synthetic construct
  source 1..40: mol_type = other DNA
acgaacgaaagctctgagctctcacatgcttggcttcacg

SEQ ID NO: 13 - DNA, 23 bp, synthetic construct
  source 1..23: mol_type = other DNA
attcatatgctctaaccttgagt

SEQ ID NO: 14 - DNA, 20 bp, synthetic construct
  source 1..20: mol_type = other DNA
tgggcgatcttctccagcag

SEQ ID NO: 15 - DNA, 20 bp, synthetic construct
  source 1..20: mol_type = other DNA
acacctacaccttcgtctcc

SEQ ID NO: 16 - RNA, 19 nt, synthetic construct
  source 1..19: mol_type = other RNA
gcacgaacagcttccactc